(19) United States
(12) Patent Application Publication
(10) Pub. No.: US 2003/0114673 A1
(43) Pub. Date: Jun. 19, 2003

(54) 2-AMINO-5,6-DIHALO-3,4-DIHYDROQUINAZOLINES WITH BLOOD PLATELET REDUCING PROPERTIES

(76) Inventor: Philip C. Lang, Toms River, NJ (US)

Correspondence Address: Delio & Peterson, 121 Whitney Avenue, New Haven, CT, 06510, US

(21) Appl. No.: 10/148,296

(22) PCT Filed: Nov. 29, 2000
(86) PCT No.: PCT/GB00/04561

(51) Int. Cl. A61K 31/517; C07D 239/72

(52) U.S. Cl. 544/292; 514/266.4

(57) ABSTRACT

Compounds of formula (E) (wherein X and Y, which may be the same or different, each represents Cl, Br or F) and tautomers thereof have been found to have enhanced platelet count reducing properties. Also provided are methods for synthetically making the compounds of formula (E) and pharmaceutically acceptable addition salts thereof and a method of reducing the platelet count in patient by administering to the patient a platelet reducing effective amount of a compound of formula (E) preferably together with a pharmaceutically acceptable carrier. A pharmaceutical composition is also provided which contains a compound of formula (E) as the active ingredient together with pharmaceutically acceptable excipients. Further provided is a compound of formula (E) for use in therapy and the use of a compound of formula (E) for preparing a medicament for reducing platelet count in a patient.

This invention relates to certain 2-amino-5,6-dihalo-3,4-dihydroquinazolines of use in the treatment of thrombocythemia, secondary to myeloproliferative diseases, such as Essential Thrombocythemia (ET), Polycythemia Vera (PV), Chronic Myelogenous Leukemia (CML) and Other Myeloproliferative Diseases (OMPD).

Anagrelide, which is chemically 6,7-dichloro-1,5-dihydroimidazo-[2,1-b]-quinazolin-2(3H)-one, is indicated for the treatment of patients with thrombocythemia, secondary to myeloproliferative diseases, such as Essential Thrombocythemia (ET), Polycythemia Vera (PV), Chronic Myelogenous Leukemia (CML), and Other Myeloproliferative Diseases (OMPD).

The formula for Anagrelide is:

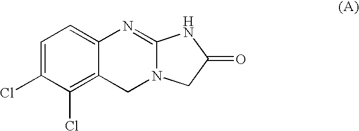

The major clinical action of Anagrelide is to decrease and maintain the platelet count within normal limits. The most common adverse effects associated with the use of Anagrelide are related to its vasodilatory and positive inotropic effects. Cardiovascular side effects associated with the use of Anagrelide have included vasodilation (<5%), tachycardia (7.5%), palpitations (26.1%), and congestive heart failure (1.5%).

A study by Gaver et al. (1981) “Clinical Pharmacology and Therapeutics”, 29, 381-392, demonstrated that Anagrelide was extensively metabolized by human subjects to a minimum of at least four to five compounds excreted in the urine and that peak plasma concentrations of the parent drug were only 6 ng/mL. It was reported that very little of the Anagrelide dose (<1%) was excreted as unchanged drug in the urine. These studies raised the possibility that the thromobocytopenia observed with Anagrelide may be due to a metabolite rather than the parent drug. In the Gaver study the use of ¹⁴C-anagrelide indicated by HPLC a minimum of four to five metabolites which accounted for over 75% of the urinary radioactivity. It was concluded that the antiaggregation activity observed in humans is possibly related to the presence of Anagrelide and an active metabolite(s).

U.S. Pat. No. 3,932,407 and its Reissue Patent No. Re. 31,617, which patents are hereby incorporated by reference, disclose Anagrelide type compounds of the formula:

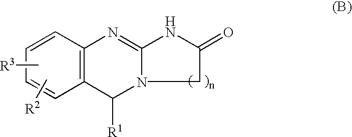

in which R¹ is H, phenyl or lower alkyl, R² and R³ when alike are H, chloro, bromo, fluoro, lower alkyl, hydroxy or lower alkoxy, R² and R³ when different are H, chloro, bromo, fluoro, SO₃H, CF₃, hydroxy, nitro, amino, phenyl, lower alkyl of 1 to 3 carbon atoms or lower alkoxy of 1 to 3 carbon atoms, or when taken together R² and R³ are methylenedioxy or the residue of phenyl ring, and n is an integer of 1 or 2; and pharmaceutically acceptable acid addition salts thereof. The compounds, which are disclosed as hypotensive, blood platelet reducer and/or bronchodilator agents, are prepared inter alia by a multistep process ending in the reaction of CNBr with an ethanolic solution of a compound of the formula:

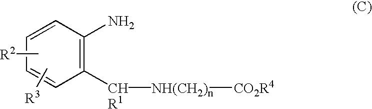

in which R¹, R², R³ and n are as described above, and R⁴ is lower alkyl.

Anagrelide may be prepared directly from a lower alkyl-N-(6-amino-2,3-dichlorobenzyl) glycine of Formula (C) by reaction in an alcoholic solution with CNBr. U.S. Pat. No. 4,146,718 incorporated herein by reference, discloses an improved process whereby higher yields of Anagrelide may be obtained by reacting a compound of Formula (C) with, for example, CNBr, CNCl or CNI in an inert, aprotic organic solvent and isolating the novel intermediate of Formula D:

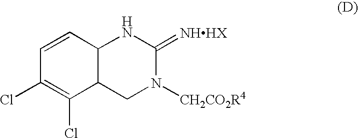

wherein R⁴ is (lower) alkyl and X is chloro, bromo or iodo. In a preferred embodiment X is bromo and R⁴ is methyl, ethyl, n-propyl, isopropyl or n-butyl. In a more preferred embodiment X is bromo and R⁴ is methyl, ethyl or n-propyl. In a most preferred embodiment X is bromo and R⁴ is ethyl.

Intermediate compound (D) is then treated with a base to produce the Anagrelide compound of Formula A.

As reported in U.S. Pat. No. 4,146,718, supra, although the compounds of Formula (D) are primarily intended as intermediates in the preparation of Anagrelide, they themselves have blood platelet antiaggregative properties.

It is an objective of the present invention to provide alternative compounds having enhanced platelet reducing properties.

A further object of the invention is to provide a method for reducing the platelet count in a patient by administering a platelet reducing amount of a compound having enhanced platelet reducing properties preferably with a pharmaceutical carrier in a unit dose.

It is yet another object of the present invention to provide a pharmaceutical composition containing a compound effective for reducing the platelet count together with pharmaceutically acceptable excipients.

According to one aspect of the present invention there are provided compounds of the formula (E)

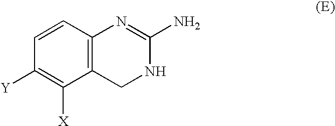

(wherein X and Y, which may be the same or different, each represents Cl, Br or F) and tautomers thereof.

The compounds of formula (E) may exist in tautomeric form as compounds of formula (F)

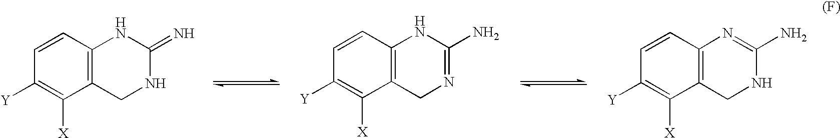

The invention also extends to pharmaceutically acceptable acid addition salts of compounds of formula (E) and their tautomers.

If the compounds of formula E and their tautomers are initially obtained as acid addition salts, these may be converted into the corresponding free dihydroquinazolines according to procedures well know to those skilled in the art. The free dihydroquinazolines may themselves be converted into different acid addition salts by reaction with inorganic or organic acids. Suitable acids include, for example, hydrochloric, hydrobromic, sulfuric, phosphoric, fumaric, succinic, lactic, citric, tartaric, maleic and methanesulfonic acids.

In a preferred embodiment Y and X are both Cl and the compound is 2-amino-5,6-dichloro-3,4-dihydroquinazoline.

Viewed from another aspect, the invention provides a method for making a compound of the formula:

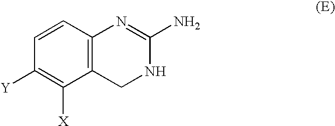

in the form of an HZ addition salt which comprises reacting a compound of formula (K)

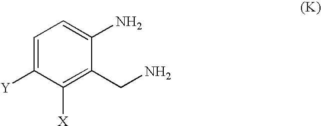

(wherein X and Y are as defined in claim 1) with a compound of formula CNZ (wherein Z is Cl, Br or F).

Compound (K) may, for example, be obtained by reduction of a compound of formula (J)

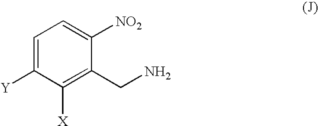

(wherein X and Y are as previously defined).

Compound (J) may, for example, be obtained by reduction of a compound of formula (I)

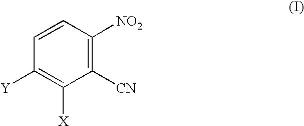

(wherein X and Y are as previously defined).

Compound (I) may, for example, be obtained by reacting a compound of formula (H)

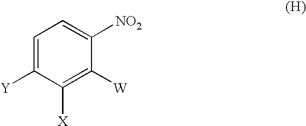

(wherein X and Y are as are as previously defined and W represents Cl, Br or F) under cyanation conditions.

Compound (H) may, for example, be obtained by nitrating a compound of formula (G)

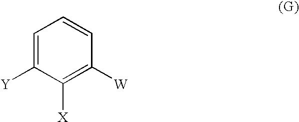

(wherein X, Y and W are as previously defined.

Compound (J) may, for example, also be obtained by aminating a compound of formula (O)

(wherein X and Y are as previously defined and V is Cl, Br or F).

Compound (O) may, for example, be obtained by sequentially hydroxylating and halogenating, preferably in in situ sequential reactions, a compound of formula (M) by way of intermediate (N)

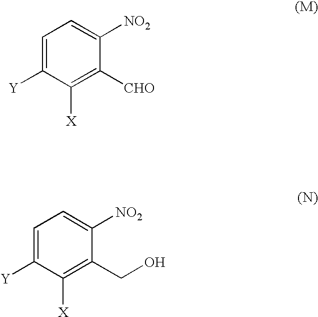

(wherein X and Y are as previously defined).

Compound (M) may, for example, be obtained by nitrating a compound of formula (L)

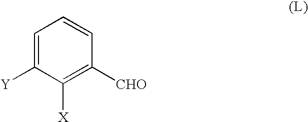

(wherein X and Y are as previously defined).

Compound (J) may, for example, also be obtained by reductive amination of compound (M).

Viewed from a further aspect, the invention provides a method for reducing the platelet count in a patient which comprises administering to the patient a platelet reducing effective amount of a compound of the formula of compound (E), preferably in combination with a pharmaceutical carrier. The compound is administered in a unit dosage form typically in the form of a capsule, tablet, enteric coated tablet, intravenous formulation or nasal spray.

Viewed from a still further aspect, of the invention a pharmaceutical composition is provided containing as the active ingredient at least one compound as in formula (E) together with pharmaceutical acceptable excipients such as mannitol and methyl cellulose. The pharmaceutical composition is in a unit dosage form which is administered to patients taking the pharmaceutical composition.

The invention also provides a compound of formula (E) for use in therapy; and the use of a compound of formula (E) for preparing a medicament for reducing platelet count in a patient.

Compounds of the formula (E)×

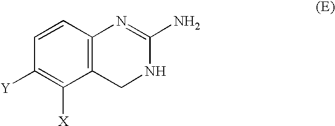

(wherein X and Y, which may be the same or different, each represent Cl, Br or F); tautomers thereof and pharmaceutically acceptable addition salts thereof have been found to have enhanced platelet reducing properties.

2-Amino-5,6-dichloro-3,4-dihydroquinazoline (compound (E) wherein X and Y are both Cl) is the preferred compound for use in reducing the platelet count in patients and is preferably provided as the hydrobromide salt which is water soluble. This is to be compared with Anagrelide hydrochloride which is nearly insoluble in water,

2-Amino-5,6-dichloro-3,4-dihydroquinazoline has been synthesized and has been found to be an active molecule for causing reduction of platelet counts, without the side effects of inotropy and haemorrhaging associated with Anagrelide. Only very mild antihypertensive activity was noticed during cardiovascular screening of the compound.

As shown below in the Examples, a preferred process is set forth for making 2-amino-5,6-dichloro-3,4dihydroquinazoline from commercially available starting materials. In one process 1,2,3-trichlorobenzene is used as the starting material and is nitrated using, for example, nitric/sulfuric acids to form 1,2,3-trichloro-4-nitrobenzene. This compound is then reacted with a cyanating agent such as CuCN to form 2,3-dichloro-6-nitrobenzonitrile. The nitrile is then reacted under reducing conditions using for example B₂H₆ to form 2,3-dichloro-6-nitrobenzylamine HCl which is then reduced and reacted with CNX to form the desired compound.

In another process, 2,3-dichlorobenzaldehyde is reacted to form 2,3-dichloro-6-nitrobenzaldehyde. The aldehyde is then reacted to form the alcohol and in Situ reacted to form the corresponding halomethyl compound. The halomethyl compound is then reacted to form the benzylamine. The benzylamine is reduced to form the diamine and the diamine reacted with CNX to form the desired compound. In another process the 2,3-dichloro-6-nitrobenzaldehyde (above) is reductively aminated to the benzylamine and the process continued to form 2-amino-5,6-dichloro-3,4-dihydroquinazoline.

### EXAMPLES

A metabolite of Anagrelide was isolated from human urine and purified by reversed-phase HPLC. Structural characterization was carried out by the use of mass spectrometry and ¹H-NMR spectroscopy. Positive liquid secondary ion mass spectrometry provided an exact mass of the protonated molecule as 216.0080 and a calculated chemical composition of C₈H₇N₃Cl₂. The 1H-NMR spectrum indicated that the compound contained two ortho coupled aromatic protons and two benzylic protons located on a quinazoline ring Capillary LC/electrospray mass spectrometry showed that the metabolite was a homogeneous single compound which gave an intense protonated molecular ion at m/z 216 with a chlorine isotope peak at m/z 218. In source fragmentation resulted in the generation of structurally significant fragment ions including the loss of a primary amino group and cleavage of the quinazoline ring. Based on these data the metabolite was identified as 2-amino-5,6-dichloro-3,4-dihydroquinazoline. The structure of the metabolite was confirmed by comparison with a synthesized sample.

Metabolism of Anagrelide was examined in a number of in vitro systems including rat and human liver microsomes, human whole blood, human bone marrow cells and rat intestinal mucosa. Surprisingly, under all of the conditions that were tried, Anagrelide was completely resistant to metabolism. It appears that in vivo metabolism occurs by a route that cannot be readily mimicked in vitro. It is hypothesized that the first step of in vivo metabolism involves hydroxylation of the carbon α to the carbonyl followed by amide hydrolysis and loss of the elements of glyoxylic acid.

In a recent pharmacokinetic and excretion study of [¹⁴C] Anagrelide to Rhesus Monkeys, no human metabolite (2-amino-5,6-dichloro-3,4-dihydroquinazoline) was detected, showing that humans uniquely metabolize Anagrelide.

### Preparation of 2-amino-5,6-dichloro-3,4-dihydroquinazoline Hydrobromide From 2,3-dichlorobenzaldehyde

Preparation of 2,3-dichloro-6-nitrobenzaldehyde

A solution of 40 g of 2,3-dichlorobenzaldehyde in 160 mL of concentrated sulfuric acid (95-98% w/w) is heated to 40° C. and stirred to form a solution, then cooled to 20-25° C. Concentrated nitric acid (69-71% w/w; 24.7 g) is added to this solution over 20 minutes (an ice bath is used to maintain a reaction temperature of 20-30° C.). The reaction mixture is stirred at room temperature for L hour, and then added in portions to 600 mL of water. The resulting suspension is stirred for 2 hours and filtered. The filter cake is washed (3×50 mL water). The filter cake is agitated with 200 mL of water for 2 hours and filtered. The filter cake is washed (3×50 mL of water) and dried in vacuo to give a mixture of the title product and the isomer, 2,3-dichloro-5-nitrobenzaldehyde

The crude product is triturated with hexane for 3 hours and filtered. The filter cake is washed with hexane (2×70 mL). This trituration procedure is repeated with fresh hexane until the 5-nitro isomer is removed. The filter cake is then dried in vacuo to give the purified title product in 44 to 50% yield.

Preparation of 2,3-dichloro-6-nitrobenzylalcohol

A solution of 40 g of 2,3-dichloro-6-nitrobenzaldehyde in 200 mL of toluene was stirred for five minutes. Then, 7.4 mL of methanol was added and mixing continued until all the solids had dissolved. Separately, a solution of 2.41 g of sodium borohydride in 120 mL of toluene was prepared. The benzaldehyde solution was added by drops to the borohydride solution over 20 minutes to maintain the reaction temperature below 25° C. The reaction mixture was stirred for 24 hours at room temperature under nitrogen. 40 mL of water was added and the mixture stirred for 15 minutes. The aqueous layer was removed and the organic layer washed with water (3×40 ML). The organic layer was azeotropically dried using a Dean-Stark trap, and concentrated to 280 mL. The 2,3-dichloro-6-nitrobenzylalcohol was used without further purification.

Preparation of 1,2-dichloro-3-chloromethyl-4-nitrobenzene

Under nitrogen, 27.9 mL of triethylamine was added to the concentrated solution of 2,3-dichloro-6-nitrobenzylalcohol prepared in the previous step. To this solution, 14.6 mL of thionyl chloride was added via an addition funnel over 15 minutes. Following addition, the solution was heated to 45-50° C. for 18 hours, then cooled to room temperature under nitrogen. Water and toluene were added to the reaction mixture and the mixture filtered. The filtrate was diluted with water, and the aqueous layer removed. The organic layer was washed with water (4×40 mL), and dried by azeotropic distillation. The solution was concentrated to give 1,2-dichloro-3-chloromethyl-4-nitrobenzene which could be used without further purification.

Preparation of 2,3-dichloro-6-nitrobenzylamine

Potassium carbonate and 2 equivalents of phthalimide are ground together and heated to reflux with 2 equivalents of 1,2-dichloro-3-chloromethyl-4-nitrobenzene for three hours. Upon cooling, the substituted benzylphthalimide crystallizes and is filtered in 60-80% yield. 2,3-Dichloro-6-nitrobenzylamine is obtained in a 75-95% yield with acid hydrolysis using hydrochloric acid from the substituted benzylphthalimide.

Preparation of 2-amino-5,6-dichlorobenzylamine

A solution of 7.131 g of tin (II) chloride dihydrate in 4.7 L of concentrated hydrochloric acid was prepared. Separately, 1.631 g of 2,3-dichloro-6-nitrobenzylamine hydrochloride was dissolved in 9.5 L of concentrated hydrochloric acid. The solution of the amine was added to the tin chloride solution over 1-2 hours to maintain the reaction temperature below 45° C. The mixture was stirred for 2 hours at 40-45° C. in a warm water bath, and cooled in an ice/methanol bath to −5° C. The mixture was filtered, and the isolated solids slurried with 13 L water, 6.5 kg ice, and 6 L methylene chloride. Sodium hydroxide (50!% aqueous solution) was added until pH>12, and the organic layer was removed. The aqueous phase was extracted with methylene chloride (2×6 L), and the combined organic layers washed with water (6 L portions) until pH 7-8. The organic layers were dried over magnesium sulfate (500 g) and charcoal (200 g) for 16 hours, and filtered. The filtrate was concentrated to an oil, and crystallized from isopropanol at −20° C. to give 2-amino-5,6-dichlorobenzylamine in 58 to 96% yield.

Preparation of 2-amino-5,6-dichloro-3,4-dihydroquinazoline

A solution of 2.58 kg of 2-amino-5,6-dichlorobenzylamine in 2.7 L of toluene was prepared at 50-60°. A solution of cyanogen bromide (1.558 kg in 8 L toluene) was added over 2-3 hours. Following addition, the reaction mixture was stirred for 1.5 hours at room temperature, then heated to reflux for 1 hour. The mixture was cooled to room temperature for 12 hours, and the mixture filtered. The filter cake was washed with toluene (2×2 L) followed by hexane (2×2 L) and dried to give 2-amino-5,6-dichloro-3,4-dihydroquinazoline hydrobromide in 70to 90% yield.

Characterization of 2-amino-5,6-dichloro-3,4-dihydroquinazoline

Elemental Status

Analysis calculated for C₈H₇N₃Cl₂·HBr: C, 32.35%; H, 2.72%; N, 14.15%; Br, 26.91%; Cl, 23.88%; Found: C, 32.78%; H, 2.73%; N, 13.91%; Br, 27.17%; Cl, 23.90%.

The above method of preparation may be shown by the following equation sequence:

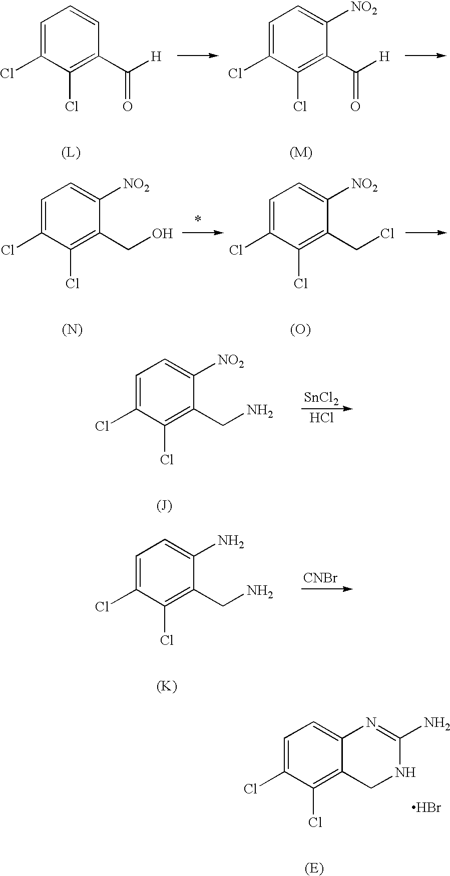

This reaction was run in situ with no isolation of the alcohol from the alcoholization reaction.

In another process of the invention the 2,3-dichloro-6-nitrobenzaldehyde formed above (compound (M) wherein X and Y are both Cl) is reductively aminated to form 2,3-dichloro-6-nitrobenzylamine (compound (J) wherein both X and Y are Cl) as follows:

A solution of the aldehyde in methanol is stored for 16 hours at room temperature with 10 equivalents of ammonium hydroxide and 0.5 equivalents of sodium cyanoborohydride. The reaction was quenched by the addition of water and 1N HCl. The product amine was isolated by concentration in a 60-84/ yield.

2,3-Dichloro-6-nitrobenzylamine is then reacted as above to form the desired 2-amino-5,6-dichloro-3,4-dihydroquinazoline hydrobromide.

### Preparation of 2-amino-5,6-dichloro-3,4-dihydryquinazoline Hydrobromie from 1,2,3-trichlorobenzene

Preparation of 1,2,3-trichloro-4-nitrobenzene

A mixture of 9.0 kg of 1,2,3-trichlorobenzene in 13.32 L of concentrated sulfuric acid is added by drops to a solution of 4.13 L of 70% nitric acid and 4.13 L of concentrated sulfuric acid at 25-30° C. The slurry is stirred for 1.5-2 hours, then poured over 35 kg of ice and filtered. The filter cake is washed (15 L water) and then dissolved in 58 L of ethyl acetate. The organic phase is washed with water, aqueous sodium bicarbonate solution, and aqueous sodium chloride solution. The solution is dried over magnesium sulfate and concentrated to give the title product.

Preparation of 2,3-dichloro-6-nitrobenzonitrile

A solution of 1.831 kg of 1,2,3-trichloro-4-nitrobenzene and 0.861 kg of copper cyanide in 1.21 L of N,N-dimethylformamide is heated to 155° C. for 2 hours, then cooled to room temperature. A solution of 3.24 kg of ferric chloride hexahydrate, 0.806 L of concentrated hydrochloric acid, and 4.87 L of water is added and the solution heated to 65° C. for 20 minutes. The mixture is cooled, stirred with 0.55 kg of charcoal and 4 L of toluene, and filtered. The organic phase is separated and the aqueous phase extracted with toluene. The combined toluene layers are washed with water and 6 N HCl, dried and concentrated to give a slurry. The slurry is dissolved in 1.5 L of methanol, and stored at 5° C. for 24 hours. The nitrile product is collected by filtration, washed with 1.5 L cold methanol and dried at 40° C.

Preparation of 2,3-dichloro-6-nitrobenzylamine HCl

A solution of 0.213 kg of 2,3-dichloro-6-nitrobenzonitrile in 1.176 L of dry tetrahydofuran is added to 1.6L of borane-tetrahydofuran complex (BH₃.THF) between 0-5° C. The solution is stirred for 2 hours at room temperature, heated to 66° C. for 2 hours, and then cooled to 15° C., before adding 0.329 L of cold methanol. The mixture is held for 17 hours and evaporated under vacuum to an oil. The oil is dissolved in ethyl acetate, cooled to 0° C. and sparged with hydrogen chloride gas. The salt is collected by filtration, washed with cold ethyl acetate, and dried.

As shown above, the nitro group in the above compound is then reduced with SnCl₂/HCl to form 2,3-dichloro-6-nitrobenzylamine HCl (compound (K) wherein both X and Y are Cl) and then reacted with CNBr to make the desired 2-amino-5,6-dichloro-3,4-dihydroquinazoline hydrobromide.

The above reaction sequence starting from 1,2,3-trichlorobenzene is as follows:

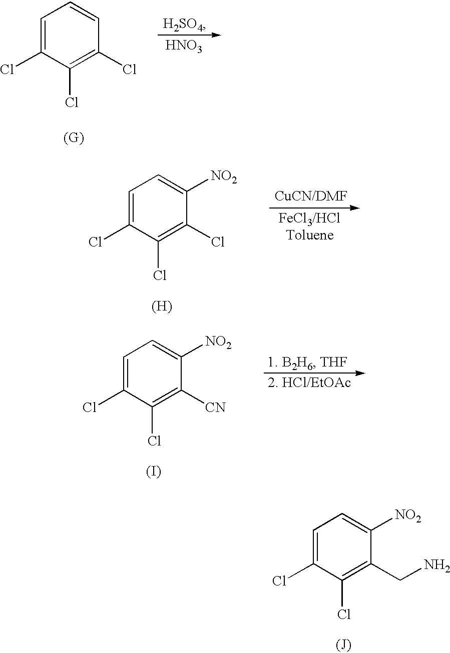

The effectiveness of 2-amino-5,6-dichloro-3,4-dihydroquinazoline hydrobromide. of the invention is shown wherein daily intraperitoneal injection of the preferred compound in 6- to 8-month old mice resulted in a dose-dependent decrease in circulating platelet levels. Administration of 100 μg/day of the preferred compound was sufficient to decrease platelet counts within 24-48 hours stabilizing to 50% of normal by day 5. Even at high doses (300 μg), the preferred compound did not alter white blood cell counts, bleeding time or lead to any apparent signs of toxicity or haemorrhaging. A profound inhibition of megakaryocytic endoreplication was achieved at doses 500 times less than those reported for Anagrelide, However, the was no effect on the CD15⁺ myeloid lineage suggesting that even in vitro the preferred compound selectively affects megakaryocytic lineage, but unlike Anagrelide, the compound did not inhibit platelet aggregation even at high concentrations (10 μg/mL).

While the present invention has been particularly described, in conjunction with a specific preferred embodiment, it is evident that many alternatives, modifications and variations will be apparent to those skilled in the art in light of the foregoing description It is therefore contemplated that the appended claims will embrace any such alternatives, modifications and variations as falling within the true scope and spirit of the present invention.

1. A compound of the formula: 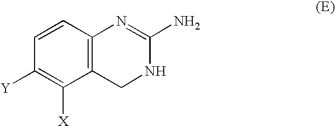
(wherein X and Y, which may be the same or different, each represents Cl, Br or F) and tautomers thereof.

2. A compound as claimed in claim 1 wherein both X and Y are Cl.

3. A pharmaceutically acceptable addition salt of a compound as claimed in claim 1 or claim 2.

4. A salt as claimed in claim 3 being the HBr salt.

5. A method for making a compound of the formula: 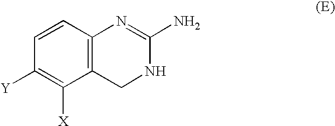
in the form of an HZ addition salt which comprises reacting a compound of formula (K) 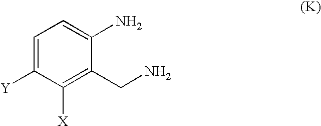
(wherein X and Y are as defined in claim 1) with a compound of formula CNZ (wherein Z is Cl, Br or F).

6. A method as claimed in claim 5 wherein the compound of formula (K) is obtained by reduction of a compound of formula (J) 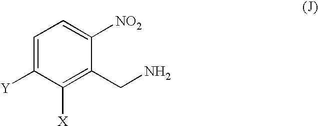
(wherein X and Y are as defined in claim 1).

7. A method as claimed in claim 6 wherein the compound of formula (J) is obtained by reduction of a compound of formula (I) 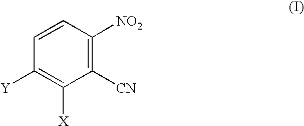
(wherein X and Y are as defined in claim 1).

8. A method as claimed in claim 7 wherein the compound of formula (I) is obtained by reacting a compound of formula (H) 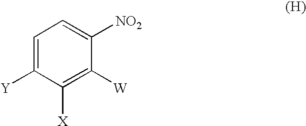
(wherein X and Y are as claimed in claim 1 and W represents Cl, Br or F) under cyanation conditions.

9. A method as claimed in claim 8 wherein the compound of formula (H) is obtained by nitrating a compound of formula (G) 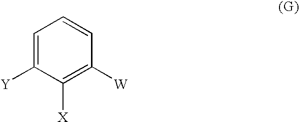
(wherein X and Y are as defined in claim 1 and W is as defined in claim 8).

10. A method as claimed in claim 6 wherein the compound of formula (J) is obtained by aminating a compound of formula (O) 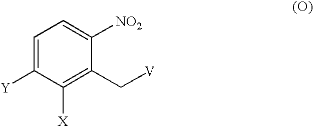
(wherein X and Y are as defined in claim 1 and V is Cl, Br or F).

11. A method as claimed in claim 10 wherein the compound of formula (O) is obtained by sequentially hydroxylating and halogenating a compound of formula (M) 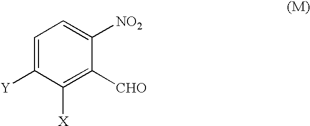
(wherein X and Y are as defined in claim 1).

12. A method as claimed in claim 11 wherein the compound of formula (M) is obtained by nitrating a compound of formula (L) 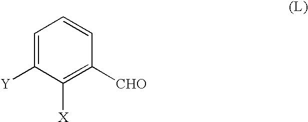
(wherein X and Y are as defined in claim 1).

13. A method as claimed in claim 6 wherein the compound of formula (J) is obtained by reductive amination of a compound of formula (M) as defined in claim 11.

14. A method of reducing platelet count in a patient which comprises administering to said patient a platelet reducing effective amount of a compound as claimed in any of claims 1 to 4.

15. A pharmaceutical composition containing as the active ingredient at least one compound as claimed in any one of claims 1 to 4 together with one or more pharmaceutically acceptable excipients.

16. A compound as claimed in any one of claims 1 to 4 for use in therapy.

17. Use of a compound as claimed in any one of claims 1 to 4 for preparing a medicament for reducing platelet count in a patient.
